(19) United States
(12) Patent Application Publication
(10) Pub. No.: US 2010/0260725 A1
(43) Pub. Date: Oct. 14, 2010

(54) Materials and Methods for Treating Allergic and Inflammatory Conditions

(76) Inventors: Shyam S. Mohapatra, Lutz, FL (US); Xiaoyuan Kong, Tampa, FL (US); Weidong Xu, Tampa, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/679,630

(22) PCT Filed: Sep. 24, 2008
(86) PCT No.: PCT/US08/11113

§ 371 Date: Jun. 28, 2010

Related U.S. Application Data
(60) Provisional application No. 60/974,668, filed on Sep. 24, 2007.

(51) Int. Cl. A61K 35/12; A61P 11/00

(52) U.S. Cl. 424/93.7

(57) ABSTRACT

The subject invention provides for the utilization of bone-marrow derived stem cells in the treatment of allergic and inflammatory diseases. In one embodiment, the invention provides for treatment of asthma. Bone-marrow derived stem cells can be used for decreasing inflammation and alter the course of immune response in the lung.

### CROSS-REFERENCE TO RELATED APPLICATION

The present application claims the benefit of U.S. Provisional Application Ser. No. 60/974,668, filed Sep. 24, 2007, which is hereby incorporated by reference herein in its entirety, including any figures, tables, nucleic acid sequences, amino acid sequences, and drawings.

### BACKGROUND OF THE INVENTION

According to the American Lung Association, lung diseases are one of the top three causes of death in America. Lung diseases, like asthma, afflict about 6.2 million children under 18 years of age, and 20.5 million adults. Based on current estimates, asthma causes approximately $11.5 billion in medical costs, and up to $16.1 billion when lost productivity is included.

Asthma is a chronic lung disease characterized by persistent inflammation caused by infiltrating eosinophils and/or neutrophils. Furthermore, T cells particularly T-helper type 2 (Th2) and T-helper type 1 (Th1) cells may contribute to the inflammation by increasing cytokine concentrations. Cytokines have been linked to perpetuating and amplifying the asthmatic inflammatory response. Current corticosteroid treatments target the increased and abnormal expression of cytokines in airway cells.

Th1 cells have been shown to produce IL-12 and IFN-g, whereas Th2 cells produce IL-4, IL-5, IL-10 and IL-13. These cytokines, IL-4, IL-5, IL-9 and IL-13, are specific to allergic inflammation and derive from Th2 cells. Early studies using an IL-4 antagonist, altrakincept, evidence the usefulness of a steroid-replacing agent in moderately severe asthma (Borish et al., 1999). IL-5 is discussed by Scott Greenfeder and colleagues (Greenfeder et al., 2001). IL-5 is specific to eosinophilic inflammation and airway hyperresponsiveness (AHR). Anti-IL-5 antibody (mepomizulab) has shown a great deal of efficacy reducing eosinophils in the circulation and the airways. Curiously, mepomizulab treatments has not reduced allergen response or in AHR (Leckie et al., 2000; Kips et al., 2000). IL-9 has been less intensively investigated than the other Th2 cytokines, but appears to amplify Th2-cell-mediated responses (Zhou et al., 2001). Th2 cytokines are likely important in the pathophysiology of allergic diseases, including asthma.

The reason Th2 cells are more prominent than Th1 cells in asthma is still unknown, however a popular theory suggests infection and exposure to endotoxins in dirt may alter the balance between Th1 and Th2 cells. Genetic polymorphisms may predispose an individual to an imbalance between Th1 and Th2 cells, including single nucleotide polymorphisms (SNPs) of the endotoxin receptor CD14. Allergic asthmatics exhibit a dominant Th2 responsiveness and Th1 response is considered protective. This has important therapeutic implications and suggests that stimulating Th1 cells might suppress Th2 cells and allergic inflammation. Alternatively, new evidence indicates natural killer T cells (NKT) may be involved in the induction of asthma, either acting as an effector cell for asthma alone or inducing Th1 and Th2 cells (Meyer et al., 2006).

Adult stem cells (ASCs) are relatively undifferentiated cells. A subset of these ASCs, called side-population (SP) cells, possesses a lineage-negative phenotype with enriched long-term culture capability and unlimited self-renewal, which typically requires interaction with other cells in the microenvironment referred to as a niche. SP cells have been identified in hematopoietic compartments of mice, humans, monkeys, and swine and in nonhematopoietic tissues including skeletal muscle, brain, and lung. These SP cells can be distinguished from their more differentiated counterparts by a characteristic Hoescht profile, which can be used to isolate the cells by dual-wavelength flow cytometry using this ability to efflux fluorescent Hoechst 33342 dye, a process mediated by the ATP-binding cassette (ABC) transporter proteins. Although Hoechst is able to enter live cells, it is actively pumped out by the ABC transporters p-glycoprotein, ABC3 and ABCG2 in human cells. These transporters may also be specifically inhibited by calcium channel blockers, verapamil and reserpine.

Typically, ASCs proliferate infrequently relative to that of other cells (progenitor cells) possessing proliferative capacity within the tissue. Proliferation of the stem cell results from the depletion of other proliferative cells within the tissue and leads to replenishment of the progenitor cells. For long-term maintenance of the stem cell, its proliferation must be accompanied by at least one of the progeny retaining the stem cell character of its parent. The differentiation potential of a tissue stem cell and the range of progenitor cells that may be generated are largely governed by the cellular and anatomic complexity of the tissue in which it resides. Progenitor cells that participate in the maintenance and repair of injured lung epithelium have been described for tracheobronchial, bronchiolar, and alveolar compartments (Evans et al., 1978a; Evans et al., 1978b; Evans et al., 1986). SP cell location has a functional affect on differentiation potential, with SP cells challenged with repopulating a different anatomical compartment possessing significantly lower repopulation capacity (Preffer et al., 2002).

Studies in rodent injury models have suggested the existence of endogenous lung tissue stem cells following chemical or physical depletion of progenitor cells (Borthwick et al., 2001; Giangreco et al., 2002; Hong et al., 2001; Kim et al., 2005). Three distinct regions of the lung including intercartilaginous regions of tracheobronchial airways (Borthwick et al., 2001), neuroepithelial bodies (NEB) in bronchioles (Hong et al., 2001), and the bronchoalveolar duct junction (BADJ) appear to harbor lung stem cells (Giangreco et al., 2002; Kim et al., 2005). While acute lung injury may be repaired through the endogenous stem cells, in chronically injured lungs these cells are either nonexistent or non-functional.

Stem cell therapy is being intensively investigated as a novel and potentially highly effective treatment for a wide variety of human conditions from cancer to cardiovascular disease (Abdallah and Kassem, 2008; Aejaz et al., 2007). Over the past decade, much progress has been made in developing adult stem cells as multipotent therapeutic tools capable of tissue repair and replacement of damaged cells. Adult stem cells are readily available, well-characterized, and their use avoids the ethical and bureaucratic problems that have hampered the adoption of embryonic stem cells as the cell of choice for regenerative medicine (Denker, 2006; Roccio et al., 2008).

Adult stem cells are found in virtually every tissue in the body and act as a biological reservoir for replacing worn out or damaged blood cells, skin, muscle, liver and fat cells and epithelial cells among others (Granero-Molto et al., 2008; Nomura et al., 2007; Ramos and Hare, 2007; Shi et al., 2006; Theise and Krause, 2002). Mesenchymal stromal cells (MSCs) are located primarily in the bone marrow (BM) and like other stem cells are capable of self replication (Brooke et al., 2007). Hematopoietic stem cells (HSCs) also reside within the BM, and the BM-MSCs are necessary for maintaining the proliferative capacity of the HSCs. In addition to this local function, however, MSCs are able to exit from their compartment in the BM in response to appropriate signals and travel via the bloodstream to other organs. Upon mobilization from the BM and recruitment to a specific tissue, MSCs are able to differentiate into muscle, cartilage, bone, or adipose cells (Porada et al., 2006). The relative role of circulating BM stem cells in comparison to that of stem cells resident in various organs with respect to tissue regeneration is controversial and still being elucidated. There is some evidence from animal studies that resident stem cells can handle the routine cell replacement functions, but in times of greater injury the BM stem cells may be recruited to aid in the regeneration process (Anjos-Afonso et al., 2004). MSCs are able to migrate to sites of injury and it is thought that a combination of adhesion molecules and chemokine receptors is responsible for the homing activity (Chamberlain et al., 2007).

In the lung there is a pool of stem cells that provides the progenitors for replacing cells during normal turnover, but when tissues are damaged by physical injury or chronic lung disease, additional stem cells may be required. Lung inflammation is a major cause of damage and remodeling in allergic and asthmatic conditions (Broide, 2008), while diseases such as emphysema and chronic obstructive pulmonary disease may result from cigarette smoking or inhaled particulates (Curtis et al., 2007). Other progressive diseases of the lung such as idiopathic pulmonary fibrosis have no identifiable cause, but can result in severe loss of lung function or death. Chronic lung inflammation, if untreated can cause increased matrix deposition, fibrosis, and loss of bronchiolar flexibility and alveolar function. Inhaled corticosteroids are the most frequently used treatment for inflammatory conditions and, while they do reduce eosinophilia and mucus production, they do not affect the underlying cellular and molecular causes of chronic disease. The inability to eliminate the causes of progressive lung pathology and to repair the damage to the airway and alveolae condemns the patient to an inevitably worsening condition and greater dependence on drugs with their adverse side effects.

Adult stem cell transplantation is already routinely used (at least in Europe and Asia) for treating myocardial infarction (MI), stroke and peripheral artery disease. Double-blind, placebo-controlled trials have shown that autologous BM-derived stem cells can increase left ventricular function and reduce infarct size in MI patients (Janssens, 2007). Patients in clinical trials are being given stem cells to treat cardiac disease, lower limb ischemia, stroke, arthritis, diabetes, multiple sclerosis, Alzheimer's and Parkinson's disease (Abdallah and Kassem, 2008; Aejaz et al., 2007; Brooke et al., 2007; Granero-Molto et al., 2008; Porada et al., 2006). While migration of BM stem cells to the lung has been reported (Rankin, 2008), there have been no studies evaluating the effects of transplantation with BM-MSCs on allergic lung inflammation.

While multiple signaling pathways play roles in pathogenesis of asthma, recent studies demonstrated that endogenous peptide hormones, such as the atrial natriuretic peptide (ANP), play a critical role in controlling inflammatory status of the lung. For example, U.S. Pat. No. 5,911,988 provides a treatment for asthma by administering anti-SCF (stem cell factor) antibodies. After atrial natriuretic peptide binds to its receptor NPRA, ligand-receptor complexes are internalized, processed intracellularly, and sequestered into subcellular compartments. Binding of ligand to NPRA triggers a complex array of signal transduction events and accelerates the endocytosis (Pandey et al, 2005).

### BRIEF SUMMARY OF THE INVENTION

The present invention concerns materials and methods for treating allergic and inflammatory diseases of the lung, such asthma, by bone marrow stem cells (BMSCs). BMSCs have become important in tissue repair, but their role in reducing lung inflammation has not previously been studied. BMSCs were injected into ovalbumin (OVA)-sensitized and challenged mice and the treated mouse lungs compared to non-cell injected mice for inflammation and cytokine profile and compared to non-sensitized controls.

Utilization of bone-marrow derived stem cells in asthmatic treatment is disclosed herein. Bone marrow cells express the receptor for ANP, NPRA, which evidences that bone-marrow derived stem cells can be used to decreasing inflammation and alter the course of immune response in the lung. Further, these cells can be targeted using NPRA as the receptor for endocytosing peptides and DNA into the cells.

In another embodiment of this invention, the expression of the atrial natriuretic peptide (ANP) receptor, NPRA, was identified in bone marrow-derived stem cells and lung cells after purification of these cells by Sca1+Beads and flow cytometry analysis using antibodies to CD34 and to NPRA. These results indicate that NPRA can be used as a marker of stem cells and it can also be used to target these cells for genetic modification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are graphs of RBC-depleted bone marrow. Wild type (WT) C57BL/6 bone marrow cells were incubated in the presence (FIG. 1B) or absence (FIG. 1A) of verapamil 50 μM for 15 min at 37° C. prior to staining with Hoechst 33342 (5 μg/ml) for 90 min at 37° C. The side population determined by flow cytometry is shown in the gate.

FIGS. 2A and 2B are graphs of RBC-depleted bone marrow. NPRA-KO bone marrow cells were incubated in the presence (FIG. 2B) or absence (FIG. 2A) of verapamil 50 μM for 15 min at 37° C. prior to staining with Hoechst 33342 (5 μg/ml) for 90 min at 37° C. The side population determined by flow cytometry is shown in the gate.

FIG. 3 shows a graph of the optimization of real-time PCR for NPR-1. Real time PCR detection of NPR mRNA relative expression levels are shown for the lungs of NPRA-KO mice (Npr1 KO) and WT C57BL/6 mice.

FIG. 4 shows a graph of the optimization of real-time PCR for NPR-1. Real time PCR detection was performed on NPR-1 mRNA levels in the lungs of NPRA-KO mice (Npr1 KO) and WT C57BL/6 mice. Dissociation curves of Npr1 and β-actin replicons were calculated.

FIG. 5 shows a graph of the optimization of real-time PCR for NPR-1. Real time PCR detection of NPR-1 mRNA levels in the lungs of NPRA-KO mice (Npr1 KO) and WT C57BL/6 mice show relative expression of Npr1 levels in Sca1-positive and Sca1-negative lung cells.

FIG. 6 shows a graph of real-time Q-PCR assay of stem cell markers on Sca1+ and Sca1− lung cells. Total RNA was extracted and residual genomic DNA removed using the RNAqueous-Micro Kit from Ambion.

FIG. 7 shows a graph of SuperArray analysis of lungs of NPRA-KO and WT C57BL/6 mice. The X-ray films were scanned and the spots analyzed using SuperArray Software. The relative expression level was determined by comparing the signal intensity of each gene in the array after normalization to the signal of the housekeeping gene.

FIG. 8 depicts a graph of serum cytokine levels in BALB/c mice treated with BMSCs. Mice were sacrificed at 1 week after cell injection and their blood were collected and serum have been used for cytokines measurement by BIO-PLEX Luminex system.

FIG. 9 depicts a graph of serum cytokine levels in BALB/c mice treated with BMSCs. Mice were sacrificed at 2 weeks after cell injection and their blood were collected and serum have been used for cytokines measurement by BIO-PLEX Luminex system.

FIG. 10 depicts a graph of serum cytokine levels in BALB/c mice treated with BMSCs. Mice were sacrificed at 3 weeks after cell injection and their blood were collected and serum have been used for cytokines measurement by BIO-PLEX Luminex system.

FIG. 11 depicts a graph of serum cytokine levels in C57BL/c mice treated with BMSCs at 1 week after BMSC administration. Mice were sacrificed at 1 week and their blood were collected and serum have been used for cytokines measurement by BIO-PLEX Luminex system.

FIG. 12 depicts a graph of serum cytokine levels in C57BL/c mice treated with BMSCs at two weeks after BMSC administration. Mice were sacrificed at 2 weeks and their blood were collected and serum have been used for cytokines measurement by BIO-PLEX Luminex system.

FIGS. 13A and 13B depict plots of expression of NPRA expression in lung stem cells. Cells were collected using anti-SCA-1 FITC microbeads, unstrained (FIG. 13A) or stained with SCA-1 (FIG. 13B) and analyzed by flow cytometry.

FIGS. 14A and 14B depict plots of expression of NPRA expression in lung stem cells. Cells were collected using anti-SCA-1 FITC microbeads, unstrained (FIG. 14A) or stained with anti-CD34-PE (Biolegend) (FIG. 14B) and analyzed by flow cytometry.

FIGS. 15A and 15B depict plots of expression of NPRA expression in lung stem cells. Cells were collected using anti-SCA-1 FITC microbeads, unstrained (FIG. 15A) or stained with NPRA-Alexa 647 (Santa Cruz) (FIG. 15B) and analyzed by flow cytometry.

FIGS. 16A and 16B depict plots of lung cells from C57BL/6 mice that were unstained (FIG. 16A) or stained with anti-Sca1 (FIG. 16B) and analyzed by flow cytometry.

FIGS. 17A and 17B depict plots of lung cells from C57BL/6 mice that were unstained (FIG. 17A) or stained with CD34-PE (FIG. 17B) and analyzed by flow cytometry.

FIGS. 18A and 18B depict plots of lung cells from C57BL/6 mice that were unstained (FIG. 18A) or stained with NPRA-Alexa 647 (FIG. 18B) and analyzed by flow cytometry.

FIGS. 19A-19D depict paraffin embedded lung section images of BALB/c mice subjected to staining with hematoxylin-eosin (HE). BALB/c lung sections OVA sensitized at one week (FIG. 19A) and two weeks (FIG. 19B) and BALB/c lung sections, OVA sensitized and treated with BMSCs at one week (FIG. 19C) and two weeks (FIG. 19D).

FIGS. 20A-20D depict paraffin embedded lung sections of C57/BL6 mice subjected to staining with hematoxylin-eosin (HE). C57/BL6 lung sections OVA sensitized at one week (FIG. 20A) and two weeks (FIG. 20B) and C57/BL6 lung sections, OVA sensitized and treated with BMSCs at one week (FIG. 20C) and two weeks (FIG. 20D).

FIGS. 21A-21C: Embryonic stem cells reduce lung histopathology and inflammatory cytokines. (FIG. 21A) Mice were sensitized and challenged with ovalbumin (OVA) then injected with embryonic stem cells or vehicle. At 1 and 2 weeks after ESC injection, lungs were removed, stained with hematoxylin and eosin and photographed. ESC transplantation resulted in less lung damage. (FIG. 21B) Lung homogenates were assayed by ELISA for IL-4, IL-5, TNF-alpha and IFN-gamma. ESC treatment significantly reduced IL-4 level after 1 week, and IL-4, IL-5 and TNF-alpha at 2 weeks. *p<0.05.

FIGS. 22A-22F: Total bone marrow transplantation reduces lung histopathology. Bone marrow cells were incubated with (FIG. 22B) or without verapamil (FIG. 22A) then exposed to the dye Hoechst 33342. Flow cytometry measurement of side-population (stem cells) cells was performed using simultaneous measurement with blue and red emission filters. Mice were either naïve (-OVA) (FIG. 22D) or OVA sensitized and challenged (FIG. 22C), then injected with total bone marrow cells isolated from EGFP-transgenic donor mice. Two weeks after injection, lungs were sectioned and examined for GFP fluorescence. Bone marrow cells migrated to the inflamed lung but not the healthy one. Mice were OVA sensitized and challenged then injected with total bone marrow cells. Lungs were removed 2 weeks after transplantation, stained with hematoxylin and eosin and photographed (FIGS. 22E and 22F).

FIGS. 23A-23C: Bone marrow stem cells prepared from EGFP-transgenic mice express stem-cell biomarkers. Adherent BM cells from EGFP mice were cultured on 8-well slides, fixed and stained for CD44, CD90 and Sca-1 using phycoerythrin-tagged (red) antibodies. The slides were examined by fluorescence microscopy and representative photographs are shown.

FIGS. 24A-24C: Mesenchymal stem cells migrate to the inflamed lung and reduce lung histopathology. (FIG. 24A) Bone marrow cells from EGFP-transgenic mice were cultured through 4 passages with selection for adherent cells. Donor cells were injected into mice that were naïve (-OVA) or had been sensitized and challenged with OVA. Two weeks after stem cell injection, lungs were removed, sectioned and examined for GFP fluorescence. Bone marrow stem cells migrate to the inflamed lung but not to the healthy lung. (FIG. 24B) Bone marrow stem cells were prepared as in FIG. 24A, injected into OVA sensitized/challenged mice, and 2 weeks later the lungs were removed, sectioned and stained with hematoxylin and eosin. Less lung pathology was seen in mice receiving the stem cell transplants. (FIG. 24C) Mice were treated as in FIG. 24B with bone marrow stem cells from EGFP mice. Total RNA was isolated from the lungs and subjected to RT-PCR using primers specific for GFP. No GFP was seen in lungs from naïve mice.

### DETAILED DESCRIPTION OF THE INVENTION

The subject invention concerns materials and methods for treating or preventing allergic and inflammatory disease conditions. The methods of the invention comprise administering a therapeutically effective amount of bone marrow-derived stem cells (BMSC) to a person or animal in need of treatment. In one embodiment, the BMSC are cells that are autologous to the person or animal. In another embodiment, the BMSC are cells that are heterologous to the person or animal. In one embodiment, the cells are genetically modified, for example, to express or overexpress Sca1 and/or NPRA. In a specific embodiment, the BMSC express Sca1 and/or NPRA. Disease conditions contemplated within the scope of the invention include, but are not limited to, allergic rhinitis, atopic dermatitis, bronchial asthma, and food allergies. In a specific embodiment, the disease condition is asthma. In one embodiment, methods of the invention further comprise administering an ANP peptide, or a composition comprising an ANP peptide.

The subject invention also concerns a composition comprising a substantially purified population of bone marrow stem cells. In one embodiment, the BMSC are Sca1-positive and/or NPRA-positive. The BMSC can be human BMSC. In one embodiment, the BMSC comprise SP cells. The composition can optionally comprise a pharmaceutically acceptable carrier, buffer, and/or diluent. In one embodiment, the BMSC are genetically modified. In a specific embodiment, the BMSC are genetically modified to express or overexpress Sca1 and/or NPRA.

The subject invention also concerns kits comprising in one or more containers a substantially purified population of bone marrow stem cells. In one embodiment, the BMSC are Sca1-positive and/or NPRA-positive. The BMSC can be human BMSC. In one embodiment, the BMSC comprise SP cells. The kit can optionally comprise a pharmaceutically acceptable carrier, buffer, and/or diluent. In one embodiment, the BMSC are genetically modified. In a specific embodiment, the BMSC are genetically modified to express or overexpress Sca1 and/or NPRA. Kits of the invention can also optionally contain packaging information and/or instructions for use of the kit reagents in a method of the invention. Containers in a kit of the invention can be composed of any suitable material, such as glass or plastic.

The subject invention also concerns methods for reducing an inflammatory response in a person or animal by administering a therapeutically effective amount of BMSC to the person or animal. The methods of the invention comprise administering a therapeutically effective amount of bone marrow-derived stem cells (BMSC) to a person or animal in need of treatment. In one embodiment, the BMSC are cells that are autologous to the person or animal. In another embodiment, the BMSC are cells that are heterologous to the person or animal. In one embodiment, the cells are genetically modified, for example, to express or overexpress Sca1 and/or NPRA. In a specific embodiment, the BMSC express Sca1 and/or NPRA. In one embodiment, the inflammatory response is in lung tissue.

BMSCs were injected into ovalbumin (OVA)-sensitized and challenged mice and the treated mouse lungs compared to non-cell injected mice for inflammation and cytokine profile. Non-sensitized controls were also examined. Lung sections stained with anti-GFP showed that Ovalbumin sensitized/challenged BALB/c and C57BL/6 mice express GFP in bronchoalveolar epithelium 1-2 weeks after injection while non-sensitized mice do not. Sensitized BALB/c and C57BL/6 mice injected with BMSCs showed significant reduction in lung inflammation compared to mock-injected controls. The level of serum IL-12 was higher in the cell-injected group while IL-10 and IL-13 were lower. These results demonstrate that BMSCs from non-sensitized mice can reduce lung inflammation and alter cytokine levels when injected into OVA-sensitized mice. BMC injection can be used for asthma therapy. In another embodiment of this invention, the expression of the atrial natriuretic peptide (ANP) receptor, NPRA, was identified in Bone Marrow derived stem cells and lung cells after purification of these cells by Sca1+Beads and flow cytometry analysis using antibodies to CD34 and to NPRA. These results indicate that NPRA can be used as a marker of stem cells and it can also be used to target these cells for genetic modification.

“Patient” is used to describe an animal, preferably a human, to whom treatment is administered, including prophylactic treatment with the compositions of the present invention. Mammalian species that benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo bison, horses, donkey, swine, sheep, and goats; exotic animals typically found in zoos such as bear, lions, tigers, panthers, elephants, hippopotamus, rhinoceros, giraffes antelopes, sloth, gazelles, zebras, wildebeests, prairie dogs, koala bears, kangaroo opossums, raccoons, pandas, hyena, seals, sea lions, elephant seals, otters, porpoises dolphins, and whales. As used herein, the terms “subject” “host”, and “patient” are used interchangeably and intended to include such human and non-human mammalian species.

The term “bone marrow stem cell” or “BMSC” is used to refer to adult stem cells, also called somatic stem cells, isolated from the hematopoietic compartment of an organism. Specifically, the term refers to adult stems cells isolated from the bone marrow of an organism that is not a neonate or fetus.

The “therapeutically effective amount” for purposes herein is thus determined by such considerations as are known in the art. A therapeutically effective amount of the adult stem cells, bone marrow-derived stem cells, SP cells, or any combination thereof is that amount necessary to provide a therapeutically effective result in vivo. The amount of adult stem cells, bone marrow-derived stem cells, SP cells, or any combination thereof must be effective to achieve a response, including but not limited to total prevention of (e.g., protection against) and to improved survival rate or more rapid recovery, or improvement or elimination of symptoms associated with inflammatory disorders, autoimmune disorders, asthma, or other indicators as are selected as appropriate measures by those skilled in the art. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

“Administration” or “administering” is used to describe the process in which adult stem cells, bone marrow-derived stem cells, SP cells, or any combination thereof of the present invention are delivered to a patient. The composition may be administered in various ways including parenteral (referring to intravenous and intraarterial and other appropriate parenteral routes), intraperitoneal, intrathecal, intraventricular, intracisternal, intranigral, and intranasal, among others. Each of these conditions may be readily treated using other administration routes of adult stem cells, bone marrow-derived stem cells, SP cells, or any combination thereof to treat a disease or condition.

The term “essentially” is used to describe a population of cells or a method that is at least 90% purified, preferably at least 95% purified, and more preferably 98 or more % purified. Cells according to the present invention are preferably essentially free of hematopoietic cells, i.e. CD 34 positive cells fractions.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Bone Marrow Cells (BMCs) Characterization in NPRA-KO and WT C57BL/6 Mice

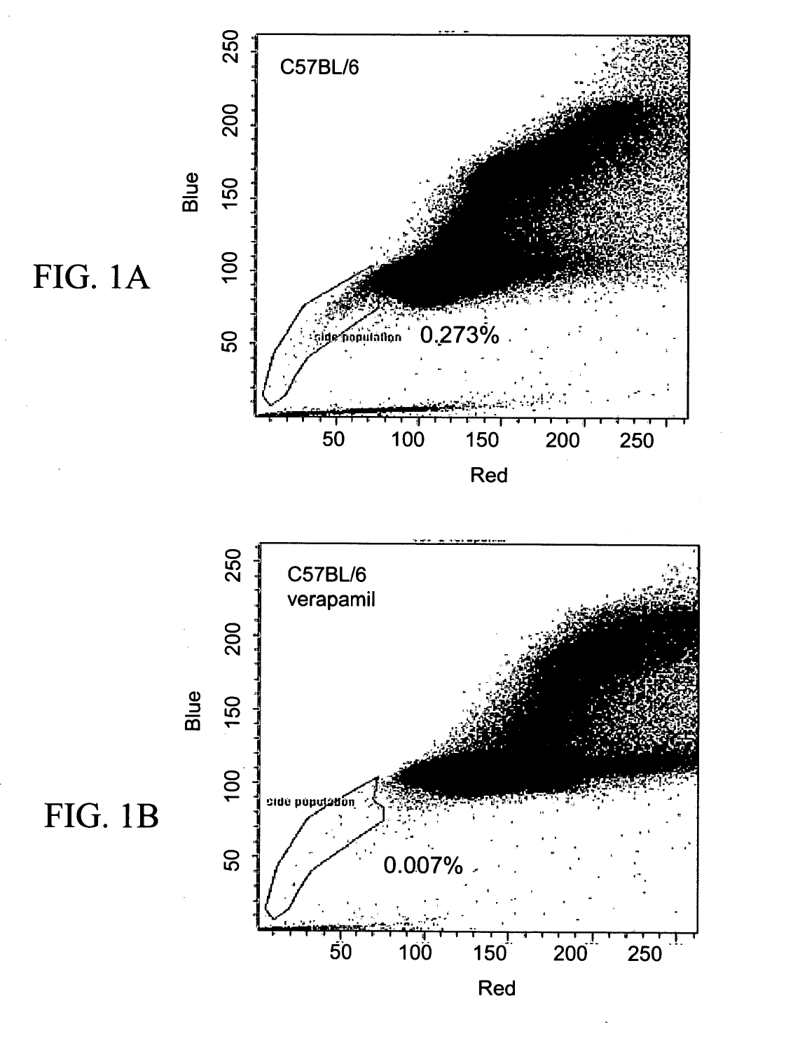

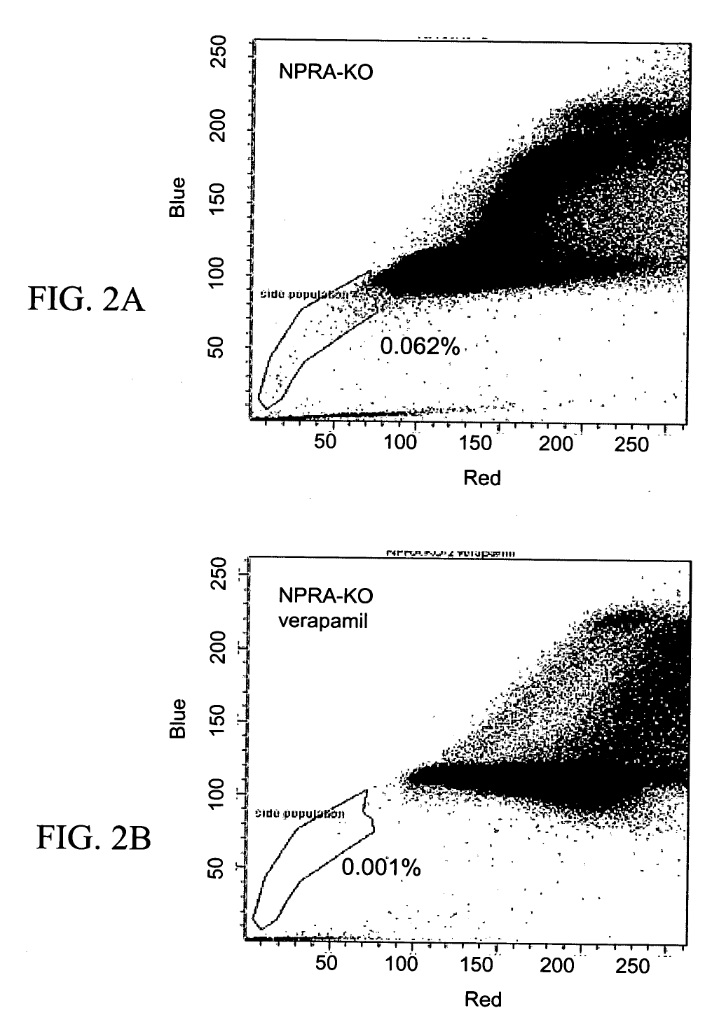

SP cells were purified using differential transport protein expression, comparing efflux patterns of certain dyes such as Hoechst and rhodamine. SP cells are found in bone marrow and normal tissues. Two wild type (WT) C57BL/6 mice and two NPRA knockout (NPRA-KO) mice were sacrificed and bone marrow cells were isolated and depleted of RBC. Cells were stained with Hoechst 33342 (5 μg/ml) in the presence or absence of 50 μM verapamil for 90 minutes at 37° C. The bone marrow SP cells were characterized by flow cytometry to determine whether there is any difference in stem cell enriched populations. WT mice showed a significantly higher percentage of SP cells (0.273%) than NPRA-KO (0.062%) mice, seen in FIGS. 1A-1B and 2A-2B. Bone marrow cells of WT mice, seen in FIG. 1A, showed a significant higher percentage of side population cells than that of NPRA-KO mice, as seen in FIG. 2A.

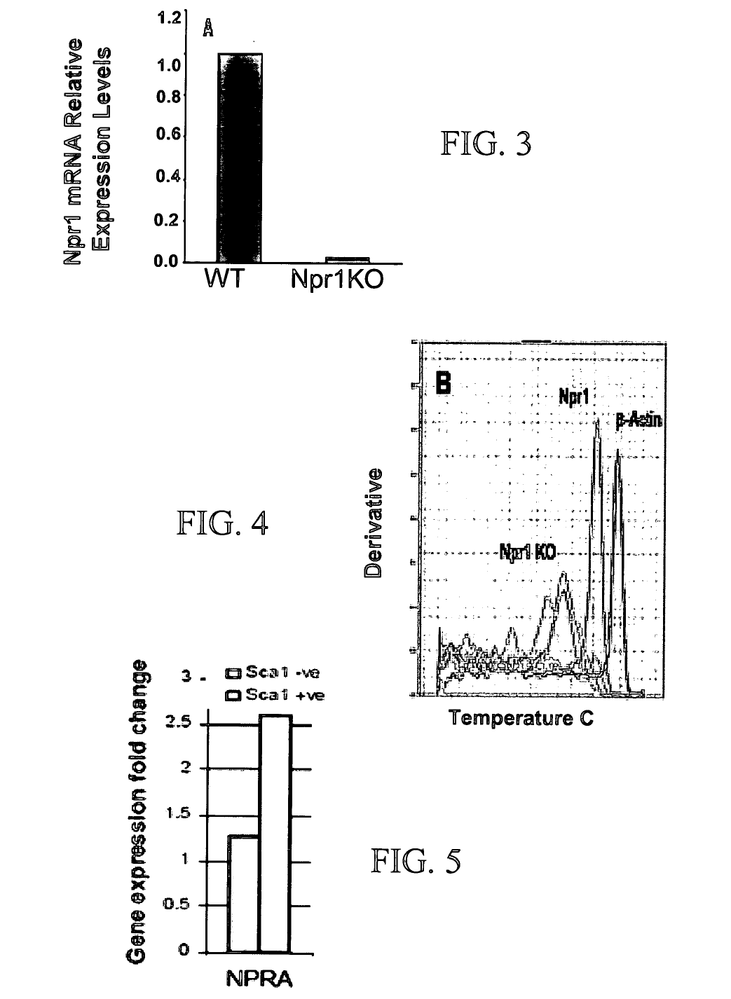

Real-time PCR detection of NPR1 mRNA was optimized using the mRNA of lung tissue of WT C57BL/6 mice. Lung mRNA of NPRA-KO mice was used as a negative control in this assay. Relative expression of Npr1 mRNA levels, seen in FIG. 3, and the dissociation curves for NPR-1 and β-actin replicons, seen in FIG. 4, were determined. The results show a single peak for NPR-1 and β-actin, representing specific amplification, while NPRA-KO (Npr1 KO) has multiple peaks at lower Tm temperature representing nonspecific binding, indicating there is no detectable NPR-1 mRNA in NPRA-KO mice.

Using this method, NPR-1 mRNA levels were compared in the Sca1-positive and Sca1-negative populations. Lung cells were isolated from sacrificed C57BL/6 mice and treated with lineage depletion (Miltenyi Biotec) to remove mature hematopoietic cells. The surviving cells were purified using SCA-1 microbeads (Miltenyi Biotec). Results suggest that Sca1-positive cells express two-fold more NPRA mRNA than Sca1-negative cells, seen in FIG. 5.

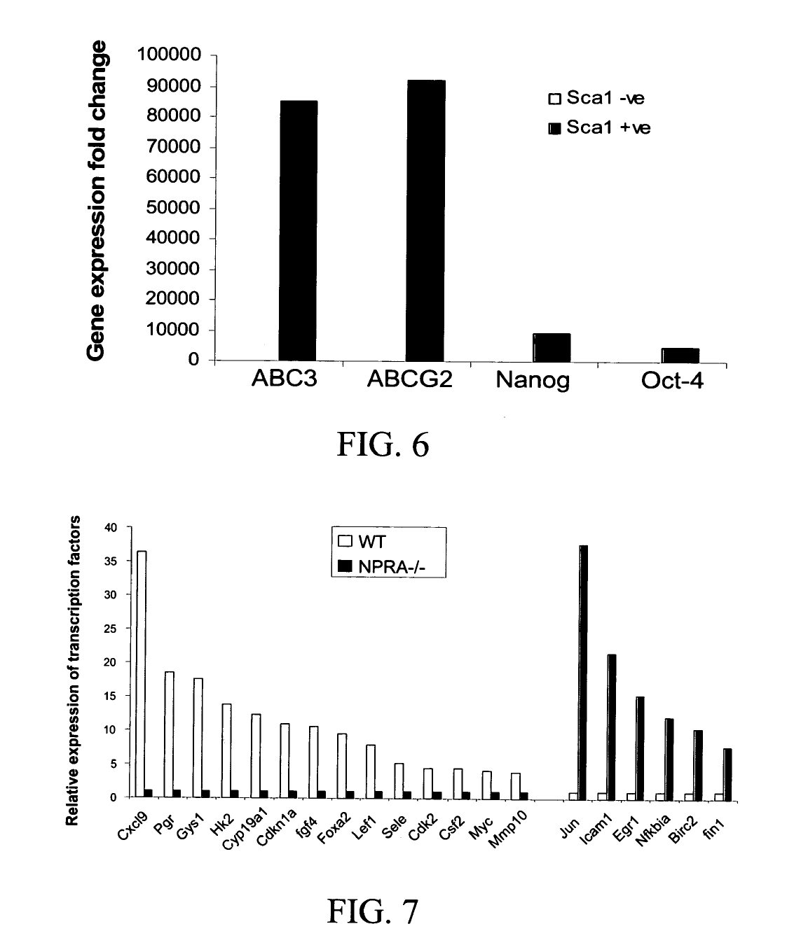

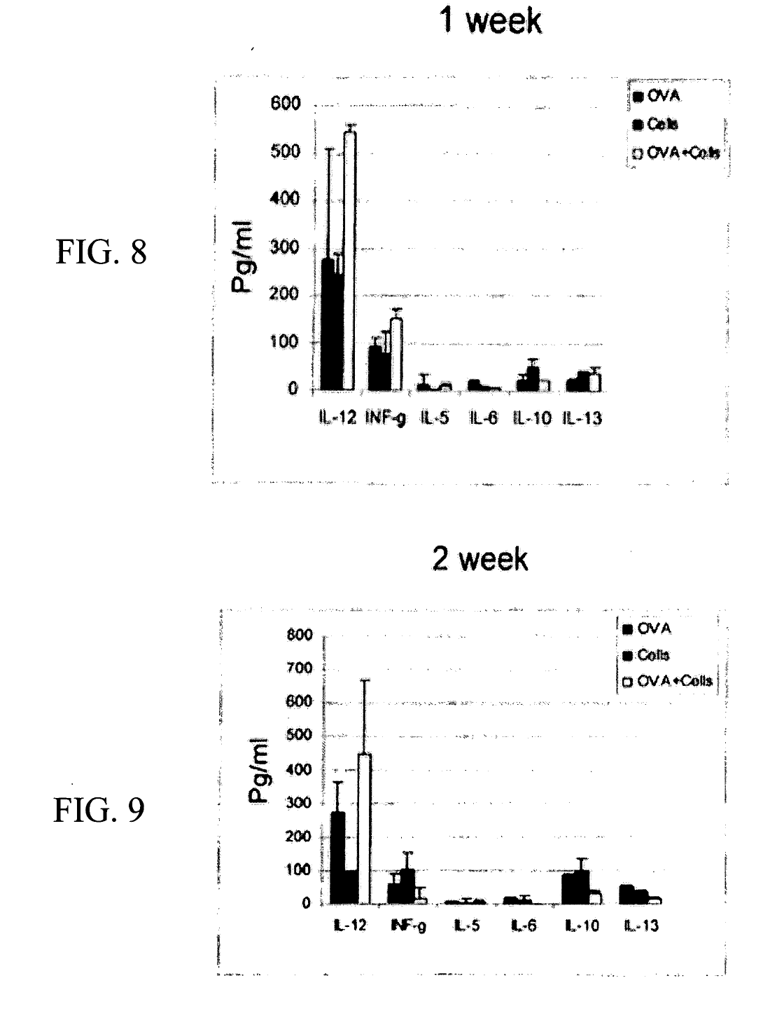

The expression of the key stem cell specific markers were next analyzed, testing ABC transporter proteins ABC3 and ABCG2, Nanog and Oct-4 from Sca1-positive and Sca1-negative lung cells. Total RNA was extracted from Sca1-positive and Sca1-negative lung cells and standard real-time quantitative PCR (Q-PCR) was performed using SYBR green and equivalent amounts of total RNA and primers specific for ABC3, ABCG2, Nanog and Oct-4. Results suggest only Sca1-positive lung cells express substantially high levels of ABC transporter proteins that play a physiologic role in detoxification, but express less Nanog and Oct-4 transcription factors than embryonic stem cells, seen in FIG. 6.

### EXAMPLE 2

### NPRA Deficiency Alters Expression of Transcription Factors

A Super Array analysis was performed on total RNA from lungs of NPRA-KO and WT C57BL/6 mice to determine whether NPRA deficiency alters expression of transcription factors. Total RNA was extracted as previously described and RNA extracts analyzed using the Oligo Gene Array Mouse Signal Transduction Pathway Finder Microarray kit (SuperArray Frederick, Md.). Results revealed that the expression of several transcription factors is significantly down-regulated or upregulated in lungs of NPRA-KO mice compared to that of WT mice. Of the 96 transcription factors on the array, 14 were expressed at significantly higher levels (>3 fold) in the lungs of wild-type mice compared to those of NPRA-KO mice, seen in FIG. 7.

Transcription factors of particular interest, elevated in WT BMSC-treated mice, include CXCL 9 (MIg), Fgf4 and FoxA2, each of which plays a role in stem cell proliferation and differentiation. CXCL9 (MIg), a member of the CXC chemokine family, is the monokine induced by interferon-gamma and is mainly produced by activated macrophages and upregulated in osteoclast precursor cells. Fgf4 regulates neural progenitor cell proliferation and neuronal differentiation and induces stem cell differentiation. FoxA2, also known as hepatocyte nuclear factor 3-β (HNF3β) plays an important role in airway epithelial differentiation and has been described as a novel tumor suppressor. Furthermore, six transcription factors including Jun, Egr1, Birc2 were significantly reduced in the lungs of WT mice compared to NPRA-KO mice. These transcription factors appear to be extremely relevant to stem cell proliferation and differentiation.

### EXAMPLE 3

### Demonstration that Bone-Marrow Derived Cells go to the Inflamed Lung of Asthmatic Mice

To induce asthma, groups (n=4) of female 4-6 week old C57BL6 or BALB/c mice were sensitized by two i.p. injections of ovalbumin (50 μg of ovalbumin in 1 mg of alum/mouse) at day 1 and 7. This was followed by three intranasal challenges on days 28, 31 and 34 with ovalbumin in saline (50 μg/mouse). Non-sensitized controls were also examined. Bone marrow stem cells (BMSCs) were collected from six to eight week old C57BL/6-TgN mice and cells were counted. 9×10⁶ BMSCs were injected into OVA sensitized and challenged BALB/c and C57BL/6 mice and control mice by tail I.V. GFP cells were confirmed by fluorescence microscope, before injecting mice by tail I.V. Two weeks later mice were sacrificed and lungs were removed and lung cryosections were stained with anti-GFP antibody to determine inflammation and cytokine profiles. Results indicate that OVA sensitized/challenged BALB/c and C57BL/6 mice express GFP in bronchoalveolar epithelium 1-2 weeks after injection while non-sensitized mice do not.

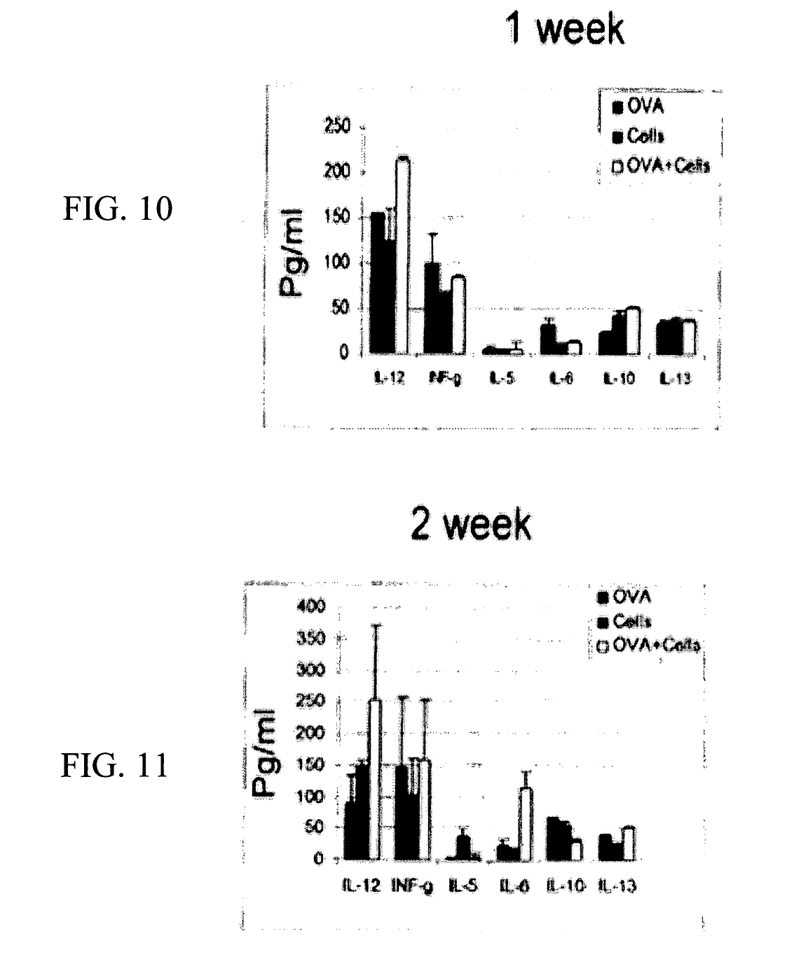

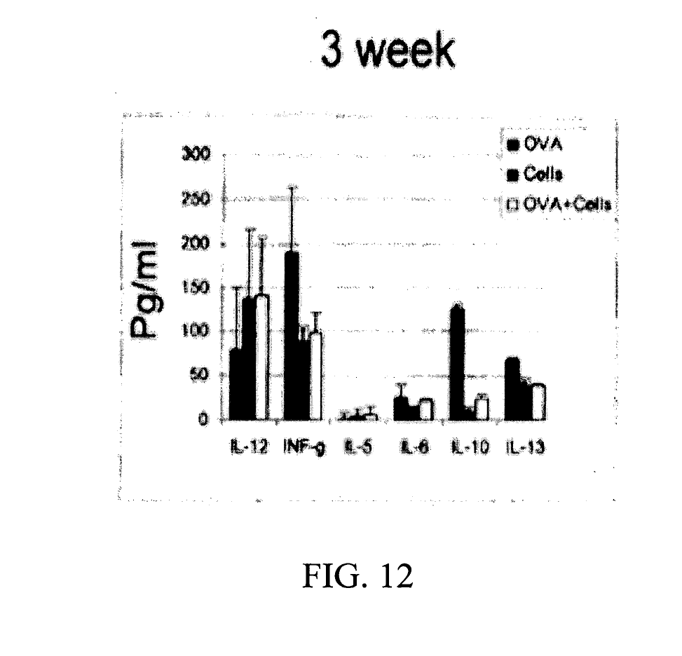

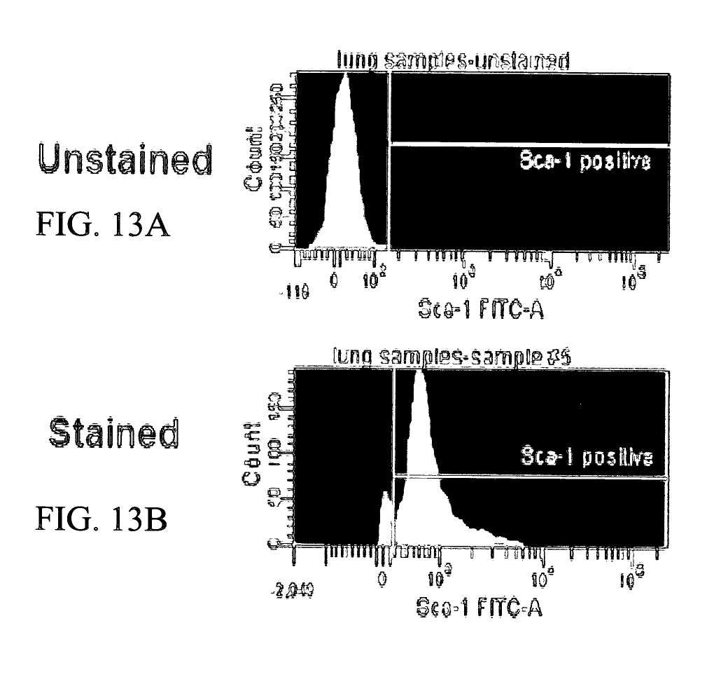

Similar experiments were run to determine plasma cytokine levels. Mice were OVA sensitized and treated with BMSCs, as described above. At 1 week or 2 weeks after BMSC treatment, the mice were sacrificed and blood collected. Serum was used for cytokine measurement by BIO-PLEX system (Bio-Rad Laboratories, Hercules, Calif.). Sensitized BALB/c and C57BL/6 mice injected with BMCs showed significant reduction in lung inflammation compared to mock-injected controls. The results indicate that syngeneic transfer of BMSCs redirects the cytokine production from Th2-type to Th1-type, as seen from increased production of Th1 promoting cytokines such as IL-12 and IFN-g and decreased production of Th-2 type cytokines such as IL-10 and IL-13, seen in FIGS. 9 and 10. Likewise, the level of serum IL-12 was elevated in the allogeneic cell-injected group while IL-10 and IL-13 were lower, seen in FIGS. 11 through 13.

These results demonstrate that BMCs from non-sensitized mice can reduce lung inflammation and alter cytokine levels when injected into OVA-sensitized mice. Further, GFP positive BMSCs were identified in the lungs of OVA-sensitized, asthmatic C57BL6 and BALB/c mice. No GFP positive cells were identified in the non-sensitized (control) mice.

### EXAMPLE 4

### Expression of NPRA in Stem Cells Derived from Bone Marrow and Lungs

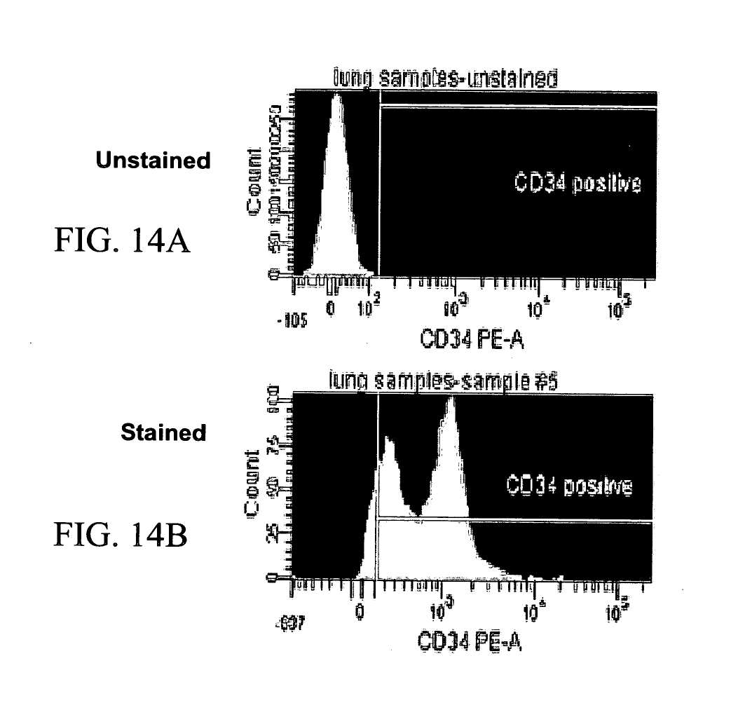

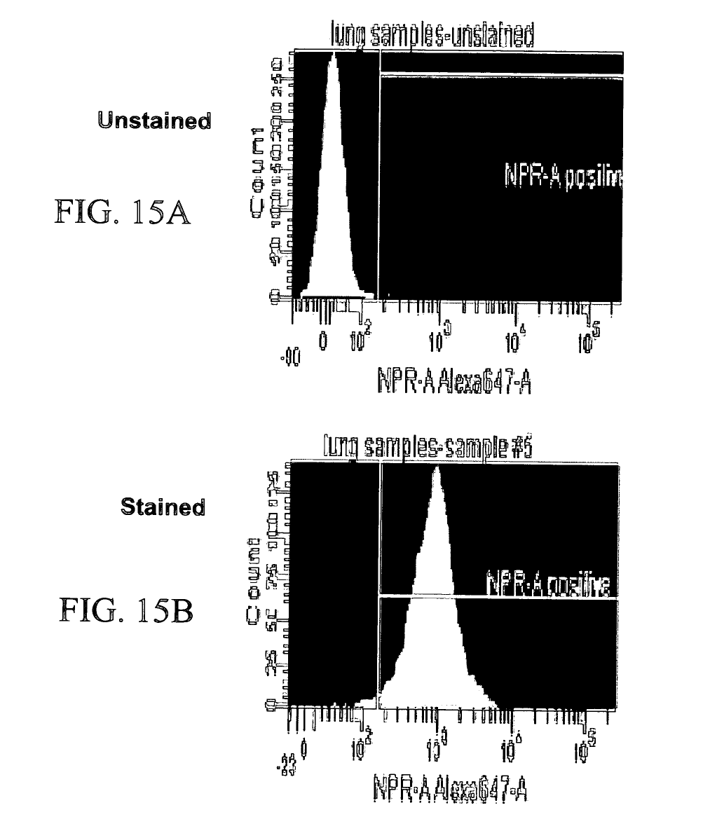

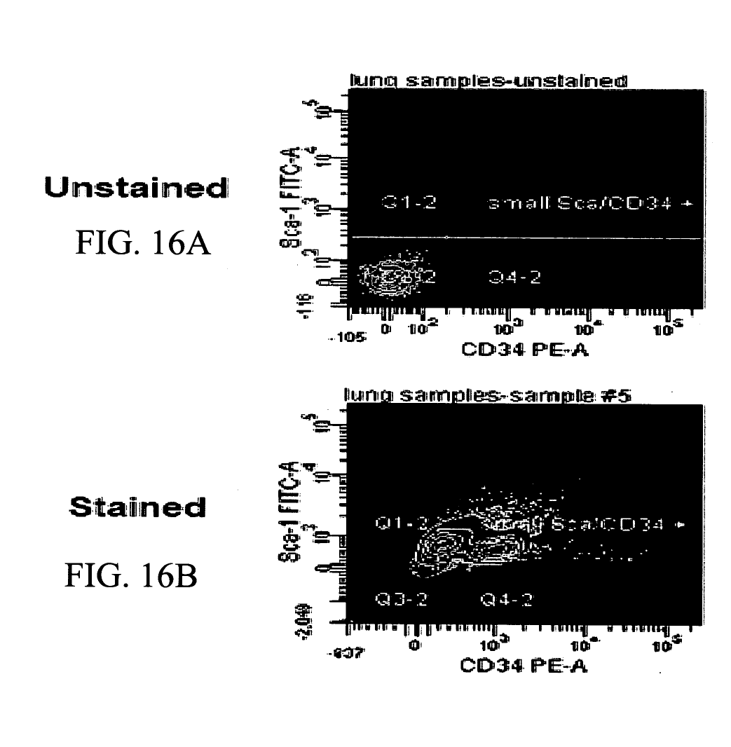

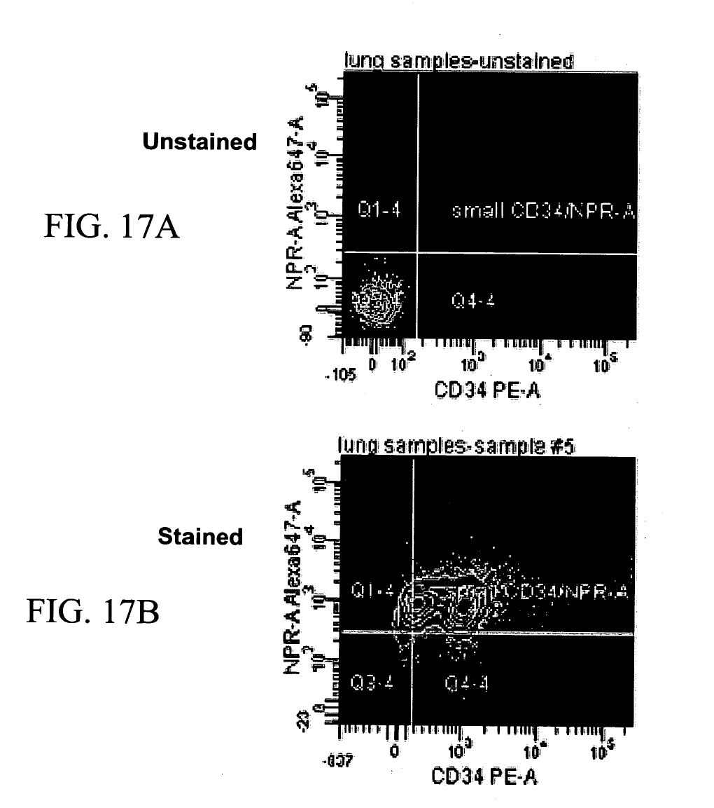

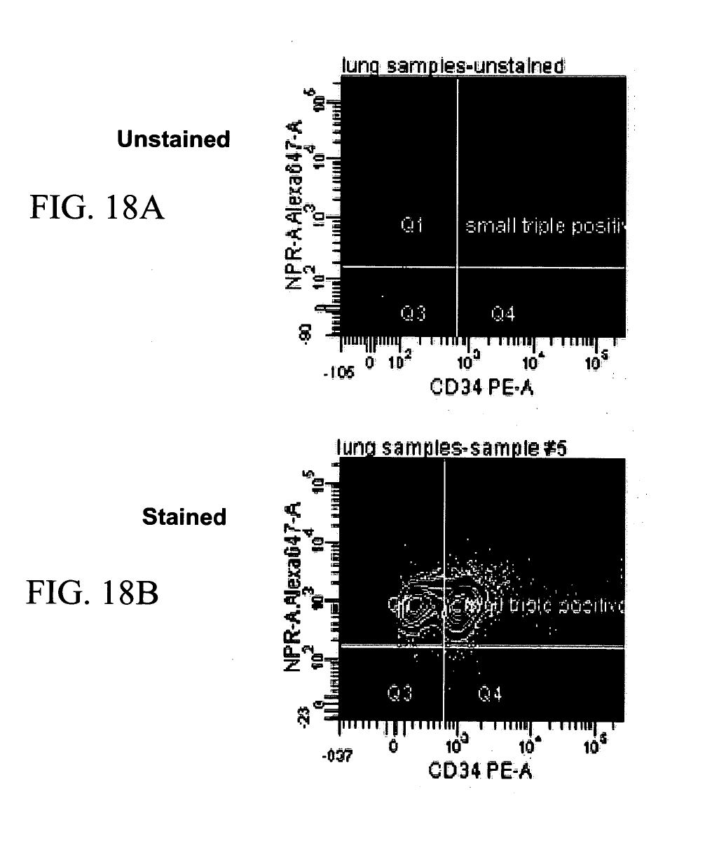

NPRA, the atrial natriuretic peptide (ANP) receptor, expression was examined in stem cells derived from bone marrow and lungs. Two ten-week old female C57BL/6 mice were sacrificed and their lungs were removed. Single lung-cell suspensions were prepared by a standard method, known in the art. A lineage cell depletion kit (Miltenyi Biotec) was used for the depletion of mature hematopoietic cells, and the lineage negative cells were collected for Sca1 selection by an anti-Sca1 FITC microbead kit (Miltenyi Biotec). Sca1-positive cells were stained with CD34-PE (Biolegend) and NPRA-Alexa 647 (Santa Cruz) antibodies and analyzed by flow cytometry. The Sca1, CD34 and NPRA expression was determined by a flow cytometry, as seen in FIGS. 14 through 19. Approximately 38% of Sca1-positive cells are also both CD34 and NPRA positive. Using a similar strategy, Sca1-positive bone marrow (BM)-derived stem cells were also examined for NPRA expression. Results showed that 35.9% of Sca1-positive BM cells are NPRA positive (data not shown). The results further show that NPR-1 can be used as a marker of stem cells and it can also be used to target these cells for genetic modification.

### EXAMPLE 5

### Demonstration that BMSCs Decrease Inflammation of the Asthmatic Lungs

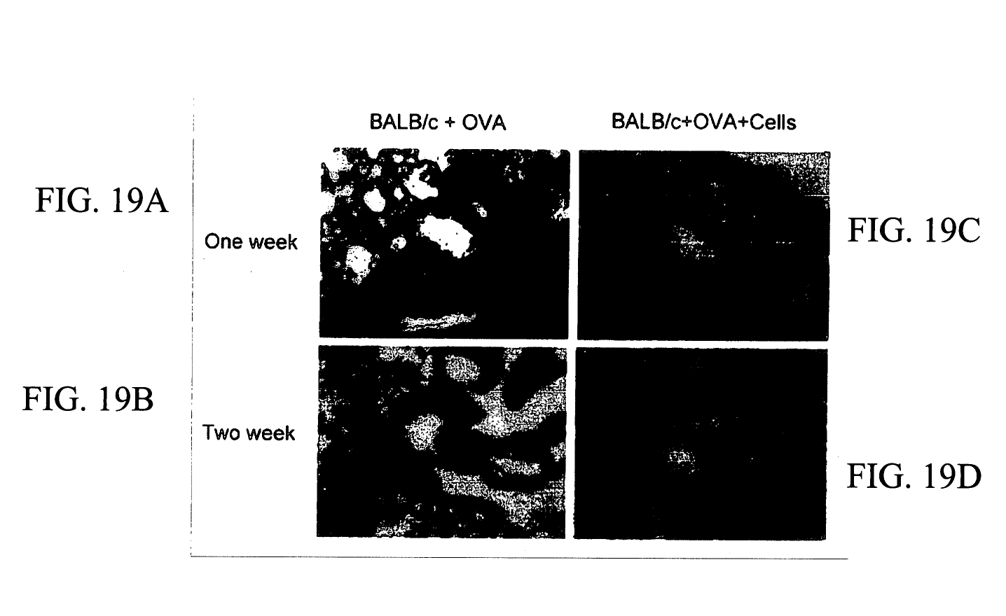

Bone marrow stem cells were collected from six to eight week old C57BL16-TgN mice and cells were counted. Female 4-6 week old C57BL6 or BALB/c mice were sensitized by two i.p. injections of ovalbumin (50 μg of ovalbumin in 1 mg of alum/mouse) at day 1 and 7. This was followed by three intranasal challenges on days 28, 31 and 34 with ovalbumin in saline. (50 μg/mouse). Non-sensitized mice served as controls. 9×10⁶ of bone marrow stem cells were injected into the OVA sensitized and control C57BL6 mice by tail I.V. to investigate syngeneic treatment. Mice were sacrificed at 1 week or 2 weeks after cell injection and their lungs were removed. Lung sections were subjected to paraffin embedding and stained with hematoxylin-eosin (HE). The results demonstrate that compared to control mice, mice treated with BMSCs showed significant reduction in inflammation up to two weeks after transfer of cells, as seen in FIGS. 19A-19D.

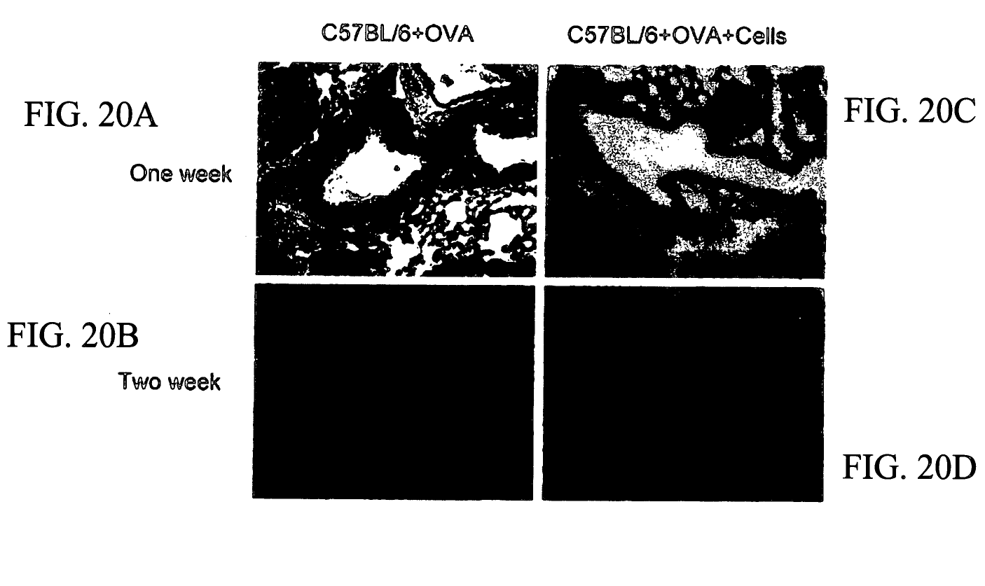

BALB/c mice and control mice were then analyzed in an identical manner to investigate allogeneic treatment. OVA sensitized and challenged mice, and controls, were administered 9×10⁶ of bone marrow stem cells by tail I.V. and the mice sacrificed as before. Lungs sections were subjected to paraffin embedding and stained with hematoxylin-eosin (HE). Compared to control mice, mice treated with BMSCs showed significant reduction in inflammation up to two weeks after transfer of cells, seen in FIG. 20A-20D.

### EXAMPLE 6

### Demonstration that BMSCs Express NPRA as a Marker

Ten week old female C57/BL6 mice were sacrificed and bone marrow cells and lung were collected and single cell suspension was prepared as described above. A Lineage cell depletion kit (Miltenyi Biotic) was used for the depletion and an anti-Sca-1 FITC microbead kit (Miltenyi Biotech) was used for Sca-1 expression. For NPRA expression, a NPRA polyclonal antibody labeled with a Zenon Alexa fluor 647 labeling kit was used. The flow cytometry data showed that both BM and lung cells have Sca-1 expression. Further, 35.9% of Sca-1 positive cells are NPRA positive in BM. 45% of Sca-1 positive cells are NPRA positive in lung cells.

To ensure that ANP-NPRa signaling pathway has effect on LSCs, NPRA expression was tested on LSCs isolated from mouse lung. CD34 and NPRA, CD34-PE (Biolegend) and NPRA-Alexa 647 (Santa Cruz) antibodies staining was performed using Sca-1 bead selected cells and stained with CD34-PE and NPRA-Alexa 647 antibodies. The Sca-1, CD34, and NPRA expression was determined by flow cytometry. There are about 38% of Sca-1 positive cells that are both CD34 and NPRA positive.

### Materials and Methods for Examples 7-10

Animals and cell line. The mouse embryonic stem cell (ESC) line SCRC-1002 (ES-C57BL/6) was purchased from ATCC (Manassas, Va.) and grown according to the supplier's instructions on a feeder layer of murine fibroblasts. The ESCs were derived from strain C57BL/6 and are germline competent. C57BL/6 mice from Jackson Labs (Bar Harbor, Me.) were used as the source of bone marrow. Mice were maintained in an AALAS-certified pathogen-free facility and handled according to standard animal use and care guidelines.

Characterization of BM-derived cell populations. Side-population (SP) cells were quantitated by flow cytometry after staining with the nuclear dye, Hoechst 33342 (BD Bioscience, San Jose, Calif.). BM cell isolates were suspended in prewarmed DMEM+5% FBS and Hoechst 33342 (200×) was added to a final concentration of 5 μg/ml. As a control, one aliquot of cells was also incubated with 50 μM verapamil which prevents the cells from excreting the dye. Cells were placed at 37° C. for 90 min to allow equilibration of the dye. After incubation, the cells were centrifuged for 5 min at 300×g at 4° C. and washed twice with cold phosphate-buffered saline (PBS). From here until flow cytometry was done, the cells were kept on ice. Just before measurement, 7-AAD was added to a final concentration of 2 μg/ml to label live cells. Readings of blue/red differential emissions were performed on a BDFacsVantage with gating for live cells and SP results are presented as percent of total BM cells.

Preparation of Adherent Stem Cells from Mouse Bone Marrow (BM). Mice were euthanized and femurs and tibias from 6 mice were used for each BM isolation. Marrow was flushed from bones with Dulbecco's modified Eagle's medium (DMEM) supplemented with 10% fetal bovine serum prewarmed to 37° C. Erythrocytes were lysed with ACK buffer (0.15 M NH₄Cl, 10 mM KHCO₃, 0.1 mM EDTA) and BM cells were collected by centrifugation at 300×g for 5 min. Cells were suspended in DMEM, counted and 2×10⁷ cells were seeded into 100 mm tissue culture dishes (BD Falcon). After two hours incubation at 37° C. in 5% CO₂/95% air, the dishes were gently rocked and nonadherent cells were pipetted off. For passaging, the adherent cells were washed three times with 10 mL of warm DMEM, recovered by a short trypsinization and counted before reseeding into additional dishes. Cells for transplant experiments were used between passages 4 and 8. The adherent population comprised about 0.02% of the total BM cells and was positive for the MSC markers Sca-1, CD90 and CD44, and negative for the HSC markers, CD34 and CD45.

Induction of allergic asthma. Mice were allergen-sensitized by i.p. injection with 50 μg of ovalbumin (OVA) mixed with 2 mg of alum adjuvant (ImJect, Pierce, Rockford Ill.). To establish an inflammatory condition in the lung, the sensitized mice were given 20 μg of OVA intranasally on two successive days prior to cell transplantation.

Transplantation with cells. Injection of cells was performed on mice that had been challenged with allergen to induce lung inflammation or on healthy naïve mice as controls. For experiments in which embryonic stem cells (ESCs) were used, cells were cultured as described above, harvested, washed with PBS, and 10⁶ cells were injected into the tail vein under anesthesia. For bone marrow transplants, 9×10⁶ total BM cells were collected as described above and injected into the tail vein. Adherent BM stem cells were cultured as described above and 10⁶ cells were injected via the tail vein.

Determination of histopathology and measurement of lung cytokine levels. At 1 and 2 weeks after injection of cells, mice were euthanized and lungs were removed. One lung was fixed, sectioned, stained with hematoxylin and eosin and examined microscopically for histopathology. The other lung was homogenized using a TissueMizer, and aliquots were analyzed for IL-4, IL-5, TNF-alpha and interferon gamma by cytokine bead array kit (BD Biosciences Pharmingen, San Diego Calif.). Unstained sections from mice injected with cells from GFP-transgenic mice were used to determine expression levels of GFP in the lung.

Examination of cells for mesenchymal stem cell markers. Adherent cells from BM taken from EGFP-transgenic mice were cultured on 8-well slides, fixed and stained with phycoerythrin-tagged anti-CD44, -CD90 and -Sca-1 for 12 h. After washing, slides were examined by fluorescence microscopy in a blinded manner by at least two persons. Additional slides were stained using PE-anti-CD45 and -CD34 to verify that these HSC markers were absent.

Determination of GFP in lung stem cells by reverse-transcriptase PCR. OVA sensitized and challenged mice were injected with adherent mesenchymal stem cells from GFP-transgenic mice, and at 1 week and 2 weeks post-injection, mice were euthanized and lungs removed. Total RNA was isolated from the lungs using the RNEasy kit (Qiagen, Valencia Calif.) and subjected to RT-PCR using primers specific for the GFP sequence (oIMR0872 and oIMR1416 from Jackson Labs, Bar Harbor Me.). PCR was performed for 35 cycles under the following conditions: denature 94° C., 2 min; denature 94° C., 30 sec; anneal 56° C., 1 min; extend 72° C., 1 min.

Statistical analysis. Student's t test was used for comparisons and p values of <0.05 were considered significant.

### EXAMPLE 7

### Embryonic Stem Cells Reduce Lung Histopathology and Inflammatory Cytokines

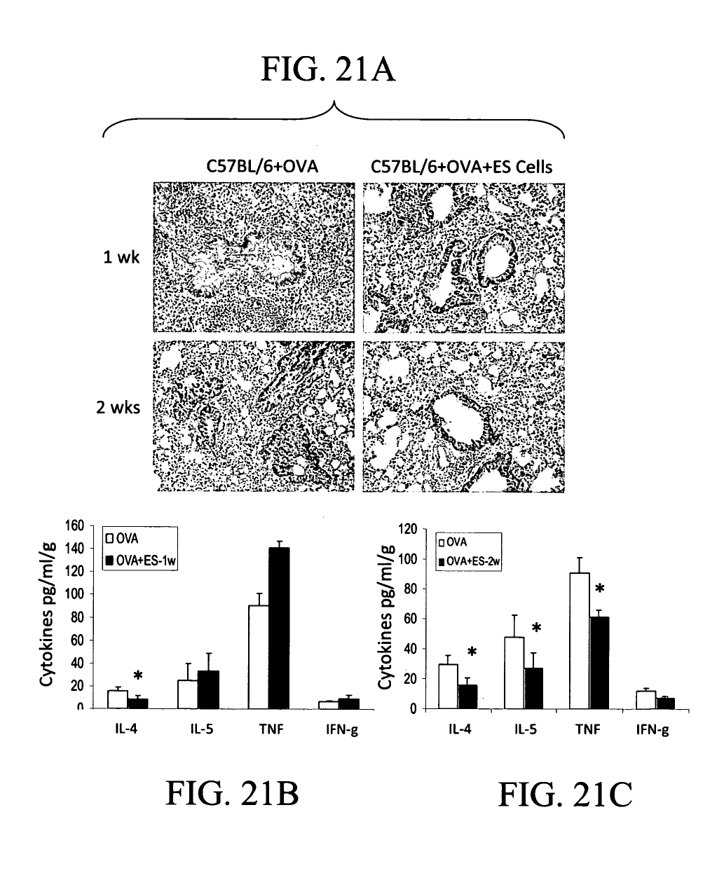

As a preliminary test of the potential of stem-cell therapy for anti-inflammatory activity in the lung we injected OVA-allergic mice with a mouse line of embryonic stem cells (ES-C57BL/6). One week after receiving ESCs, the lungs of asthmatic mice exhibited less perialveolar cellular hyperplasia and leukocyte infiltration (FIG. 21A). The anti-inflammatory activity of the ESCs was still evident two weeks later. Chronic lung disease is characterized by altered levels of a number of cytokines such as IL-4, IL-5, TNF-alpha and IFN-gamma. Lung homogenates from asthmatic mice that had been injected with ESCs had significantly less IL-4 after one week and less IL-4, IL-5 and TNF-alpha after two weeks than untreated controls. These data demonstrate that syngeneic ESCs administered by intravenous injection are capable of reducing inflammation in the lungs of asthmatic mice.

### EXAMPLE 8

### Bone-Marrow Stem Cells Migrate to the Asthmatic Lung and Reduce Inflammation

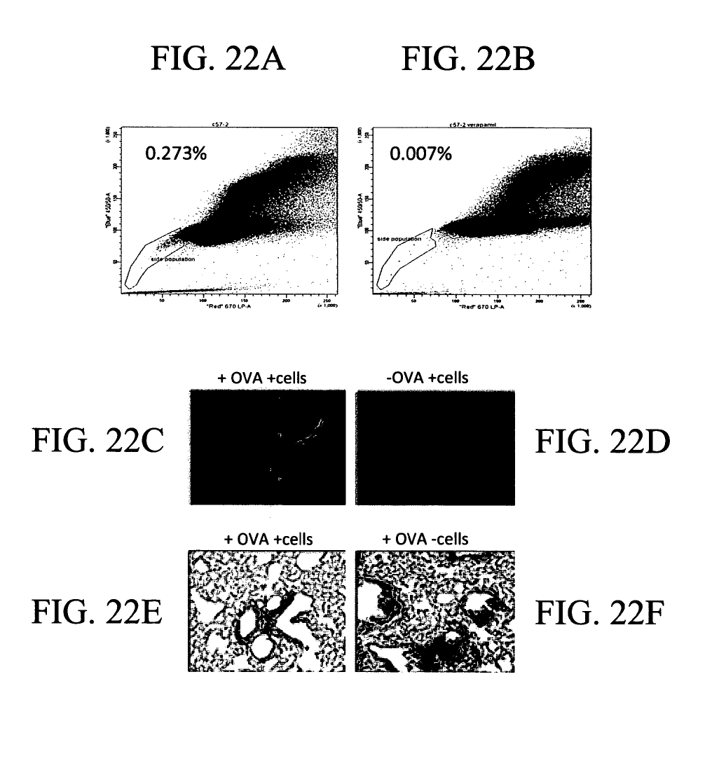

Bone marrow stem cells (BMSCs) have the advantage of being obtainable from a non-embryonic source and having consistent and well-defined properties in vitro. Thus, whole-cell, uncultured isolates of BMSCs were tested for anti-inflammatory activity in the asthmatic mouse model. To characterize the isolated cells, flow cytometry was performed after staining with the nuclear dye Hoechst 33342 which is selectively excreted by a population of progenitor cells known as side-population (SP) cells. As a control, an aliquot of the cells was incubated with verapamil which blocks the efflux of the dye. The cells were analyzed using a UV laser to excite the dye and fluorescence was measured simultaneously using blue and red filters (FIGS. 22A-22B). The percentage of the total bone marrow cells that are SP cells is seen in the small gated region in the lower left quadrant and the value shown is representative of the usual numbers obtained. As expected, verapamil treatment (FIG. 22B) blocked the dye transporter in the cells.

Total BM cells isolated from a syngeneic strain expressing green fluorescent protein (EGFP) were injected into OVA-asthmatic mice (FIG. 22C) and into healthy controls (FIG. 22D). Two weeks later, only background fluorescence was seen in the healthy mice, while the asthmatic mice with inflamed lungs exhibited strong green fluorescence. This suggests that some signals are released under inflammatory conditions that act as homing beacons for BMSCs allowing them to enter the lung and remain there for some time. Stained sections from the same lungs revealed that the BMSCs reduced lung histopathology (FIG. 22E).

### EXAMPLE 9

### Mesenchymal Stromal Cells Express Specific Stem Cell Markers

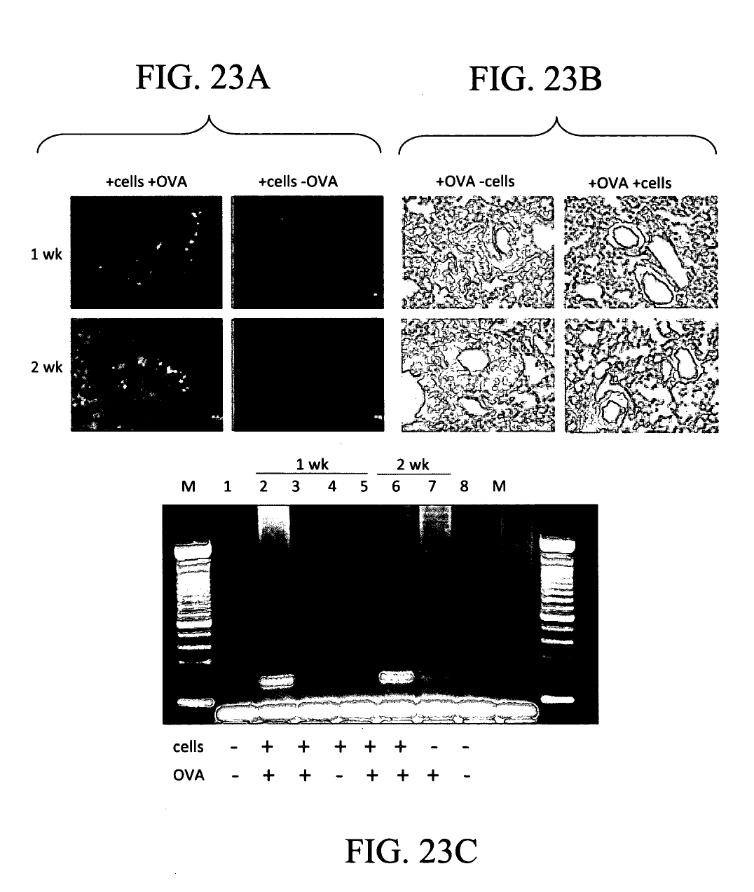

Bone marrow isolates contain a mixture of hematopoietic stem cells, mesenchymal stromal cells (MSCs) and other cells. The MSC population can be enriched by culturing the BM isolate and repeatedly rinsing off and discarding the nonadherent cells. The resulting culture consists predominantly of MSCs with few HSCs. MSCs were isolated from EGFP-transgenic mice and stained positive for the cell-surface hyaluronan receptor, CD44, the glycosylated lipid-raft protein, CD90, and stem cell antigen-1(Sca-1) (FIGS. 23A-23C). The MSCs were negative for the HSC marker, CD45 (data not shown).

### EXAMPLE 10

### MSCs Migrate to the Asthmatic Lung and Reduce Histopathology

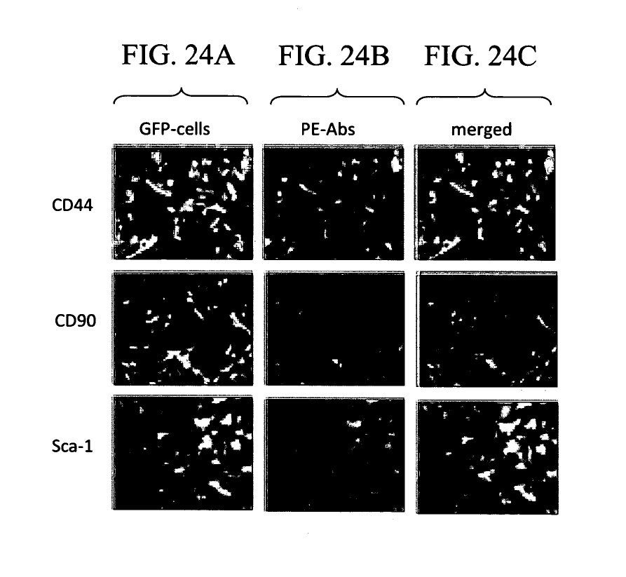

MSCs were isolated from EGFP-transgenic mice and injected into syngeneic recipients that were either sensitized and challenged with OVA or were naïve. Lungs were sectioned one week and two weeks after cell injection and examined under a fluorescence microscope. Green fluorescent cells were only seen in the lungs of asthmatic mice (FIG. 24A) confirming what was found with whole BM isolates (FIGS. 22C-22D). MSCs injected i.v. were able to home to the lung, enter the tissues and remain there. The cells reduced perialveolar cell hyperplasia and leukocyte inflammation for up to two weeks after injection (FIG. 24B).

### EXAMPLE 11

Results herein demonstrate in a mouse model that plastic-adherent, CD45-negative BM-MSCs are able to specifically home to sites of inflammation in the lung and to reduce the accompanying histopathological changes. It is well known that populations of stem cells reside within specific compartments in tissues for the purpose of regenerating lost or damaged cells, but the role of stem cells circulating in the blood as a source of cell progenitors for specific organs is still being debated. Kotton et al. (2001) reported that plastic-adherent BM cells injected i.v. into mice, migrated to inflammation sites in bleomycin-damaged lungs. The BM cells were able to engraft within the lung and to differentiate into type I pneumocytes. The engrafted cells seen in that study appeared in clusters similar to what we found in allergen-challenged mouse lungs after i.v. injection of plastic-adherent BM cells. While we did not determine differentiation, our data also show that BM-MSCs in the venous blood home to sites of inflammation in the lung and are able to repair the damage.

While several studies have shown that BM-MSCs are able to respond to injury in a specific organ and translocate to the site, the question of the relative contribution of circulating stem cells to organ maintenance and repair of tissue damage is still being debated. There is evidence from animal studies of liver regeneration that a portion of the hepatic stem cells must arise from the bone marrow (Theise and Krause, 2002) and that the degree of participation of BM-derived stem cells depends upon the severity of the damage to the liver (Anjos-Afonso et al., 2004). Injured cells may produce stromal-derived factor 1 (SDF-1) which binds to CXCR4 expressed on the surface of MSCs and acts as a homing chemokine (Ting et al., 2008). Other cytokines are likely to also play a role in mobilizing BMSCs to sites of injury and inflammation. Stem cells can also be recruited from the BM in cases of experimental cardiac infarction (Orlic et al., 2001).

Embryonic stem cells have been tested for tissue regeneration and have produced significant improvements (Janssens, 2007), but ethical considerations make it unlikely that ESCs will become a viable treatment in view of the efficacy, availability, and safety of adult SCs. Multipotent MSCs may offer a safer alternative to ESCs which have been linked to cancer formation because of their pluripotential capability. MSCs differentiate along clear lineage paths depending upon the specific signals they are exposed to and are less likely to cause problems. In our study the differentiation potential of the stem cells was not tested. The phenotype of the transplanted cells was defined according to the known stem cell markers—CD90, CD44 and Sca-1.

BM-derived MSCs have been shown to have low immunogenicity and powerful immunosuppressive activity capable of blocking both CD4+ and CD8+ T cell proliferation and CTL activation (Le Blanc and Ringden, 2007). In a study of leukemia patients with acute steroid-resistant graft vs host disease (GVDH), MSC treatment resulted in improved engraftment and lower mortality in responders (Le Blanc et al., 2008). Of particular importance was the finding that the beneficial MSC effect was the same whether HLA-matched or -unmatched donors were used. This suggests that MSCs may have sufficient immunoprivileged status that allogeneic transplants without the need for immunosuppressants are feasible. In another recent report on the use of MSCs to counteract GVDH, it was found that interferon gamma was required for the immunosuppression of T cells by infusions of MSCs (Polchert et al., 2008). Interferon gamma acted directly upon the MSCs to activate their T cell anti-proliferative properties. Given the importance of interferon gamma in lung disease, it would be of great interest to determine the potential role of interferon gamma in our observed MSC suppression of asthmatic lung inflammation.

A key factor in the potential effectiveness of MSC therapy is the ability of the cells to localize to the site of injury. Homing of other cells such as leukocytes requires a complex array of adhesion molecules, chemokines, and cytoskeletal modifiers that allows them to enter the tissue in which they are required at just the right time. Research into the mechanism of MSC homing is still in its infancy but our results demonstrate that some factors existing in the inflammatory milieu in contrast to healthy lung tissue are able to recruit MSCs from the circulation. In vitro experiments on human MSCs showed that the cells were able to respond to IL-8 by migrating across a membrane in a Boyden chamber (Mishima and Lotz, 2008). It is known that BM-MSCs express IL-8 receptors along with a panel of other chemokine receptors including CCR1, CCR7, and CCR9, CXCR4, CXCR5, and CXCR6, and the adhesion molecules ICAM-1 and ICAM-2 (Honczarenko et al., 2006). The complexity of the involved signaling pathways emphasizes the difficulty in precisely identifying the mechanism of MSC action in suppressing lung inflammation.

MSCs have been reported to block the proliferation of antigen-activated T cells through an as yet unidentified mechanism (Chen et al., 2006). The differentiation status of the cells appears to be a key factor in determining whether MSCs suppress or promote T cell proliferation. A study by Chen et al. (2007) showed that MSCs that had differentiated to chondrocytes enhanced lymphocyte proliferation and activation while the same cells in undifferentiated form were immunosuppressive. We have not examined the effects of our BM-MSCs on lymphocyte proliferation but the observed reduction in the production of the inflammatory cytokines IL-4, IL-5 and TNF-alpha would suggest at least an indirect effect on T cell activation.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 5,911,988
Borish, L C et al., 1999, “Interleukin-4 receptor in moderate atopic asthma. A phase I/II randomized, placebo-controlled trial” Am J Respir Crit Care Med.; 160:1816-1823
Borthwick et al., 2001, Am J Respir Cell Mol Biol, 24:662-670.
Chang, J C et al., 2005, Am J Respir Cell Mol Biol; 33:335-342.
Evans, M I et al., 1978a, Lab Invest, 38:648-653.
Evans, M J et al., 1986, Am J Pathol, 123:126-133.
Evans, M J et al., 1978b, Am Rev Respir Dis; 11:787-790.
Giangreco, A et al., 2002, Am J Pathol, 161:173-182.
Greenfeder, S et al., 2001, “Th2 cytokines and asthma—The role of interleukin-5 in allergic eosinophilic disease” Respiratory Research; 2:71-79.
Hashimoto, N et al., 2004, J Clin Invest; 113:243-252.
Hong, K U et al., 2001, Am J Respir Cell Mol Biol, 24:671-681.
Kim, C F et al., 2005, Cell; 121:823-835.
Kips, J C et al., 2000, “Results of a phase I trial with SCH5 a humanized anti-IL-5 antibody in severe persistent asthma [abstract]” Am J Resp Crit Care Med.; 161:A505.
Kotton, D N et al., 2005, Am J Respir Cell Mol Biol; 33:328-334.
Kotton, D N et al., 2001, Development; 128:5181-5188.
Krause, D S et al 2001, Cell; 105:369-377.
Leckie, M J et al., 2000, “Effects of an interleukin-5 blocking monoclonal antibody on eosinophils, airway hyperresponsiveness and the late asthmatic response” Lancet; 356:2144-2148.
Loi, R et al., 2006, Am J Respir Crit Care Med; 173:171-179.
Meyer, E H et al., 2006, “Glycolipid activation of invariant T cell receptor⁺NK T cells is sufficient to induce airway hyperreactivity independent of conventional CD4⁺T cells” Proc Nat Acad Sci.; 103(8):2782-7.
Ortiz, L A et al., 2003, Proc Natl Acad Sci USA; 100:8407-8411.
Pandey et al, 2005, Peptides.
Phillips, R J et al., 2004, J Clin Invest; 114:438-446.
Preffer, F I et al., 2002, Stem Cells; 20:417-427.
Zhou Y, McLane M, Levitt R C. 2001, “Th2 cytokines and asthma—Interleukin-9 as a therapeutic target for asthma” Respiratory Research; 2:80-84.
Abdallah, B. M.; Kassem, M. Human mesenchymal stem cells: from basic biology to clinical applications. Gene Ther. 15(2):109-116; 2008.
Aejaz, H. M.; Aleem, A. K.; Parveen, N.; Khaja, M. N.; Narusu, M. L.; Habibullah, C. M. Stem cell therapy-present status. Transplant. Proc. 39(3):694-699; 2007.
Anjos-Afonso, F.; Siapati, E. K.; Bonnet, D. In vivo contribution of murine mesenchymal stem cells into multiple cell-types under minimal damage conditions. J. Cell Sci. 117(Pt 23):5655-5664; 2004.
Broide, D. H. Immunologic and inflammatory mechanisms that drive asthma progression to remodeling. J. Allergy Clin. Immunol. 121(3):560-570; 2008.
Brooke, G.; Cook, M.; Blair, C.; Han, R.; Heazlewood, C.; Jones, B.; Kambouris, M.; Kollar, K.; McTaggart, S.; Pelekanos, R.; Rice, A.; Rossetti, T.; Atkinson, K. Therapeutic applications of mesenchymal stromal cells. Semin. Cell Dev. Biol. 18(6):846-858; 2007.
Chamberlain, G.; Fox, J.; Ashton, B.; Middleton, J. Concise review: mesenchymal stem cells: their phenotype, differentiation capacity, immunological features, and potential for homing. Stem Cells 25(11):2739-2749; 2007.
Chen, X.; Armstrong, M. A.; Li, G. Mesenchymal stem cells in immunoregulation.
Immunol. Cell Biol. 84(5):413-421; 2006.
Chen, X.; McClurg, A.; Zhou, G. Q.; McCaigue, M.; Armstrong, M. A.; Li, G. Chondrogenic differentiation alters the immunosuppressive property of bone marrow-derived mesenchymal stem cells, and the effect is partially due to the upregulated expression of B7 molecules. Stem Cells 25(2):364-370; 2007.
Curtis, J. L.; Freeman, C. M.; Hogg, J. C. The immunopathogenesis of chronic obstructive pulmonary disease: insights from recent research. Proc. Am. Thorac. Soc. 4(7):512-521; 2007.
Denker, H. W. Potentiality of embryonic stem cells: an ethical problem even with alternative stem cell sources. J. Med. Ethics 32(11):665-671; 2006.
Granero-Molto, F.; Weis, J. A.; Longobardi, L.; Spagnoli, A. Role of mesenchymal stem cells in regenerative medicine: application to bone and cartilage repair. Expert Opin. Biol. Ther. 8(3):255-268; 2008.
Honczarenko, M.; Le, Y.; Swierkowski, M.; Ghiran, I.; Glodek, A. M.; Silberstein, L. E. Human bone marrow stromal cells express a distinct set of biologically functional chemokine receptors. Stem Cells 24(4):1030-1041; 2006.
Janssens, S. Human embryonic stem cells for cardiac repair: the focus is on refined selection and cardiopoietic programming. Heart. 93(10):1173-1174; 2007.
Janssens, S. Stem cell therapy in acute myocardial infarction. Acta Clin. Belg. 62(5):342-347; 2007.
Le Blanc, K.; Frassoni, F.; Ball, L.; Locatelli, F.; Roelofs, H.; Lewis, I.; Lamino, E.; Sundberg, B.; Bernardo, M. E.; Remberger, M.; Dini, G.; Egeler, R. M.; Bacigalupo, A.; Fibbe, W.; Ringdén, O. Mesenchymal stem cells for treatment of steroid-resistant, severe, acute graft-versus-host disease: a phase II study. Lancet 371(9624):1579-1586; 2008.
Le Blanc, K.; Ringdén, O. Immunomodulation by mesenchymal stem cells and clinical experience. J. Intern. Med. 262(5):509-525; 2007.
Mishima, Y.; Lotz, M. Chemotaxis of human articular chondrocytes and mesenchymal stem cells. J. Orthop. Res. 2008 (in press).
Orlic, D.; Kajstura, J.; Chimenti, S.; Limana, F.; Jakoniuk, I.; Quaini, F.; Nadal-Ginard, B.; Bodine, D. M.; Leri, A.; Anversa, P. Mobilized bone marrow cells repair the infarcted heart, improving function and survival. Proc. Natl. Acad. Sci. USA 98:10344-10349.2001.
Polchert, D.; Sobinsky, J.; Douglas, G.; Kidd, M.; Moadsiri, A.; Reina, E.; Genrich, K.; Mehrotra, S.; Setty, S.; Smith, B.; Bartholomew, A. IFN-gamma activation of mesenchymal stem cells for treatment and prevention of graft versus host disease. Eur. J. Immunol. 38(6):1745-1755; 2008.
Porada, C. D.; Zanjani, E. D.; Almeida-Porad, G. Adult mesenchymal stem cells: a pluripotent population with multiple applications. Curr. Stem Cell Res. Ther. 1(3):365-369; 2006.
Ramasamy, R.; Tong, C. K.; Seow, H. F.; Vidyadaran, S.; Dazzi, F. The immunosuppressive effects of human bone marrow-derived mesenchymal stem cells target T cell proliferation but not its effector function. Cell. Immunol. 2008 (in press).
Ramos, G. A.; Hare, J. M. Cardiac cell-based therapy: cell types and mechanisms of actions. Cell Transplant. 16(9):951-961; 2007.
Rankin, S. M. Impact of bone marrow on respiratory disease. Curr. Opin. Pharmacol. 8(3):236-241; 2008.
Roccio, M.; Goumans, M. J.; Sluijter, J. P.; Doevendans, P. A. Stem cell sources for cardiac regeneration. Panminerva Med. 50(1):19-30; 2008.
Shi, C.; Zhu, Y.; Su, Y.; Cheng, T. Stem cells and their applications in skin-cell therapy. Trends Biotechnol. 24(1):48-52; 2006.
Spagnoli, A.; Longobardi, L.; O'Rear, L. Cartilage disorders: potential therapeutic use of mesenchymal stem cells. Endocr. Dev. 9:17-30; 2005.
Theise, N. D.; Krause, D. S. Bone marrow to liver: the blood of Prometheus. Semin. Cell Dev. Biol. 13(6):411-417; 2002.
Ting, A. E.; Mays, R. W.; Frey, M. R.; H of, W. V.; Medicetty, S.; Deans, R. Therapeutic pathways of adult stem cell repair. Crit. Rev. Oncol. Hematol. 65(1):81-93; 2008.

1. A method of reducing an inflammatory response in a person or animal, comprising the steps of;
administering a therapeutically effective amount of bone marrow stem cells (BMSC) to a person or animal in need of treatment; or
modulating immune cells by administering or contacting the immune cells with a therapeutically effective amount of bone marrow stem cells (BMSC).

2. The method of claim 1, wherein the bone marrow stem cells further comprise a substantially purified mononuclear cell fraction.

3. The method of claim 2, wherein the bone marrow stem cells comprise SP cells.

4. The method of claim 1, wherein the BMSC are peripherally administered.

5. The method of claim 3, wherein the bone marrow stem cells are purified using differential Sca-1 expression or NPRA expression selection.

6. The method of claim 3, wherein the SP cells are purified using differential transport of at least one dye selected from the group consisting of Hoescht and rhodamine.

7. The method of claim 1, wherein the inflammatory response is associated with asthma or allergy.

8. The method of claim 1, wherein the bone marrow stem cells express CD44, CD90, Sca1 and/or NPRA, or any combination of the foregoing, and/or do not express CD45.

9. The method of claim 1, wherein the inflammatory response is in lung tissue.

10-22. (canceled)

23. A method of treating a pulmonary disease or condition associated with an inflammatory or allergic response comprising administering a therapeutically effective amount of bone marrow stem cells (BMSC) to a person or animal in need of treatment.

24. The method according to claim 23, wherein transcription factor expression is modulated in lung cells of the person or animal.

25. The method of claim 24, wherein the transcription factor is CXCL 9 (MIg), Fgf4, or FoxA2, Jun, Egr1, or Birc2.

26. The method of claim 24, wherein the transcription factor modulates a gene encoding IL-4, IL-5, IL-10, or IL-13.

27. The method of claim 24, wherein the transcription factor modulates a gene encoding IL-12 or IFN-g.

28. The method of claim 23, wherein the bone marrow stem cells are peripherally administered.

29-30. (canceled)

31. The method of claim 23, wherein the bone marrow stem cells express CD44, CD90, NPRA and/or Sca1, or any combination of the foregoing, and/or do not express CD45.

32. The method of claim 23, wherein the BMSC are administered via a parenteral, intranasal, intrathecal, intraventricular, intracisternal, or intranigral route.

33. The method of claim 23, wherein the BMSC are autologous or heterologous BMSC.

34-35. (canceled)

36. The method of claim 23, wherein the disease or condition is asthma.

37. A composition comprising a substantially purified population of bone marrow stem cells (BMSC).

38. The composition of claim 37, wherein the BMSC are CD44-positive, CD90-positive, Sca1-positive and/or NPRA-positive, or any combination of the foregoing, and/or are CD45-negative.

39. The composition of claim 37, wherein the BMSC are human BMSC or wherein the BMSC are genetically modified.

40. (canceled)

41. The composition of claim 39, wherein the BMSC are genetically modified to express or overexpress Sca1 and/or NPRA.

42-52. (canceled)
